(19) United States
(12) Patent Application Publication
(10) Pub. No.: US 2024/0058539 A1
(43) Pub. Date: Feb. 22, 2024

(54) MULTI-COMPONENT MIXING INJECTION DEVICE

(71) Applicants: Affiliated Hospital of Qingdao University, Qingdao (CN); Qilu Hospital(Qingdao), Cheeloo College of Medicine, Shandong University, Qingdao (CN)

(72) Inventors: Zhenbo Zuo, Qingdao (CN); Jian Zhuang, Qingdao (CN); Shuining Zhao, Qingdao (CN); Jin Wang, Qingdao (CN); Bingping Zhang, Qingdao (CN)

(21) Appl. No.: 18/357,179

(22) Filed: Jul. 24, 2023

(30) Foreign Application Priority Data
Aug. 22, 2022 (CN) 2022110055575

(51) Int. Cl. A61M 5/20; A61M 5/19

(52) U.S. Cl. CPC A61M 5/2066; A61M 5/19

(57) ABSTRACT

A multi-component mixing injection device is provided. The device includes a mixer, an input end of the mixer is provided with two syringe barrels arranged side by side, an end of each of the syringe barrels is installed in a housing, and a handle is installed in the housing. The handle drives plungers to move towards the syringe barrels. The mixer includes a dynamic mixing tube, and a dynamic mixing structure is configured to mix different components in the dynamic mixing tube. The dynamic mixing structure includes multiple mixing blades, the multiple mixing blades are installed on a power output end of a transmission shaft, a power input end of the transmission shaft is connected to a direction conversion structure, and the direction conversion structure is connected to a power input structure through a synchronous belt. The device effectively improves the mixing efficiency through dynamic mixing.

### TECHNICAL FIELD

The disclosure relates to the field of marine biopharmaceutical technology, and particularly to a multi-component marine biopharmaceutical mixing injection device.

### BACKGROUND

In medical or experimental processes, it is required to use a combination of different reagents immediately after uniformly mixing. However, for many drugs, if the drugs are in a mixed state for a long time, the drugs may undergo a chemical reaction, which may affect their efficacy. Therefore, an injection device is needed that can separate different drugs and quickly mix them before injection.

A Chinese patent with an application number of CN201580033898.5 (corresponding to patent publication NO. CN106457173A) discloses a syringe for multi-component materials, a method for activating the syringe, a mixing and distribution device, and a multi-component cylinder. A static mixing element is used to achieve the mixing of multi-components, but the mixing efficiency is poor. To achieve a good mixing effect, it is necessary to extend the length of a mixer, the extension of the mixer may lead increasing of accommodation space, which also increases the waste of multi-component materials, and the extension of the mixer also reduces the accuracy of operating a needle position. In addition, it is difficult to perform a mixing operation and an injection operation of the multi-component materials with one hand, which results in poor practical convenience.

A Chinese patent with an application number of CN201520687314.3 (corresponding to patent publication NO. CN204972546U) discloses a primary and secondary type double-tube drug mix syringe, in which a single head injection head body is provided with a drug mixing chamber, the drug mixing chamber is connected to left and right syringes through a dual injection channel, and drugs from the left and right syringes are mixed in the drug mixing chamber and then are injected. The primary and secondary type double-tube drug mix syringe has a poor drug mixing effect and is difficult to perform mixing and injection actions with one hand.

A Chinese patent with an application number of CN202120870041.1 (corresponding to patent publication NO. CN215138721U) discloses a multi-component colloid static mixing connector and its injection mechanism, which achieves the mixing of multi-components through static mixing and has poor mixing efficiency per unit volume.

There are many mixing syringes used for multi-component materials in the related art, but there is a common problem of poor mixing efficiency per unit volume, and both mixing and injection actions are difficult to perform with one hand.

In summary, it is apparent that the mixing syringes in the related art have inconvenience and defects in practical use, and thus it is necessary to propose a multi-component mixing injection device to address the problem in the related art.

### SUMMARY

The disclosure provides a multi-component mixing injection device to overcome the shortcomings in the related art, which effectively improves the mixing efficiency through dynamic mixing. Under the same mixing efficiency, the size of the mixer can be minimized, the waste of a mixing medium can be reduced, and the accuracy of controlling a needle position with one hand can be improved. The operations of the mixing and injecting can be achieved with one hand, and can achieve two actions of mixing and injecting of multiple components without additional power, thereby improving convenience in use.

In order to solve the problem in the related art, the disclosure adopts the following technical solutions:
a multi-component mixing injection device is provided, which includes: a mixer; an input end of the mixer is provided with two or more syringe barrels arranged side by side, each of the syringe barrels is internally provided with a plunger, and an output end of the mixer is provided with a needle;
an end of the syringe barrel is installed in a housing, and a handle is installed to the housing and is configured to swing back and forth, a top of the handle is hinged with a pressing plate through a first rotating shaft, and the handle is configured to drive the plunger to move towards the syringe barrel through the pressing plate; and
the mixer includes a dynamic mixing tube, and a dynamic mixing structure is configured to mix different components in the dynamic mixing tube; the dynamic mixing structure includes multiple mixing blades, the multiple mixing blades are rotatably installed in the dynamic mixing tube, the multiple mixing blades are installed on a power output end of a transmission shaft, and a power input end of the transmission shaft is connected to a direction conversion structure; the direction conversion structure is connected to a power input structure through a synchronous belt; the power input structure includes an arc-shaped rack portion, and the arc-shaped rack portion is arranged on the handle.

In an embodiment, the transmission shaft includes a first transmission shaft and a second transmission shaft arranged in a coaxial manner, a power output end of the second transmission shaft is provided with the multiple mixing blade, and the second transmission shaft is in a rotary sealing connection with the dynamic mixing tube through a sealing ring.

In an embodiment, the second transmission shaft is arranged above the syringe barrels and is parallel with the syringe barrels; a power input end of the second transmission shaft is connected to a power output end of the first transmission shaft; a power input end of the first transmission shaft is connected to the direction conversion structure.

In an embodiment, the arc-shaped rack portion meshes with a first gear, a side of the first gear is provided with a second gear arranged coaxially with the first gear, the second gear meshes with a lower pulley, and the lower pulley meshes with an inner surface of the synchronous belt.

In an embodiment, the direction conversion structure includes a driving helical gear and a driven helical gear; the driving helical gear meshes with the driven helical gear; a side of the driving helical gear is provided with an upper pulley arranged coaxially with the driving helical gear, and the upper pulley meshes with the inner surface of the synchronous belt; the driven helical gear is installed on the power input end of the transmission shaft; and the driving helical gear and the driven helical gear are fixed in a left housing and a right housing through a bracket.

In an embodiment, the housing includes a left housing and a right housing arranged oppositely, the left housing and the right housing are locked by screws after being buckled together; and ends of the left housing and the right housing near the syringe barrel are provided with a buckle cover.

The handle is hinged with the left housing and the right housing through a second rotating shaft; a first pre-tightening spring is sleeved on the second rotating shaft, the first pre-tightening spring is configured to achieve automatic reset of the handle; a second pre-tightening spring is sleeved on the first rotating shaft, and the second pre-tightening spring is configured to achieve automatic reset of the pressing plate.

In an embodiment, a side of the plunger near the pressing plate is provided with a serration, and a top of the pressing plate is provided with a structure matched with the serration. In addition, angles on two sides of the serration are different, an angle on a side of the serration is small and facilitates the pressing plate moving back, and another angle of another side of the serration is large and facilitates preventing the movement of the pressing plate.

In an embodiment, an output end of the syringe barrel is an eccentric structure.

Compared with the related art, the technical solutions of the disclosure have the following advantages.

First, a mixing method in the disclosure adopts dynamic mixing instead of simple static mixing. Under the same mixing efficiency, a size of the mixer can be shortened to the minimum. The shortened mixer reduces a storage space and saves a mixing medium that needs to be filled in the storage space. At the same time, the shortening of the mixer also improves the accuracy of operating a needle position with one hand.

Secondly, the power of multi-component mixing and injection adopts a unified power source, the power source is manual and a gear set is utilized to achieve a labor-saving operation. The two operations of the mixing and injecting can be completed by one hand, thereby improving convenience in use and facilitating a medical staff to use the device.

Thirdly, an overall structural layout of the device is reasonable, which makes the device easy to be assembled and achieves a compact design. The output end of each of the syringe barrels is an eccentric structure, the eccentric structure allows the power shaft of the mixer to precisely avoid diameter positions of the syringe barrels, and thus the power shaft can be connected to the power source in a reasonable upper space.

### BRIEF DESCRIPTION OF DRAWINGS

FIG. 1 illustrates a schematic structural diagram of an embodiment 1.

FIG. 2 illustrates a schematic structural diagram of an embodiment 2.

FIG. 3 illustrates a schematic structural decomposition diagram of the embodiment 2.

FIG. 4 illustrates a structural sectional view of the embodiment 2.

FIG. 5 illustrates a schematic structural diagram of an embodiment 3 (hiding a right housing).

FIG. 6 illustrates a schematic structural decomposition diagram of the embodiment 3.

FIG. 7 illustrates a schematic diagram of a dynamic mixing structure in the embodiment 3.

FIG. 8 illustrates a partially enlarged schematic diagram of FIG. 7.

### DESCRIPTION OF REFERENCE NUMERALS

1—needle; 2—mixer; 3—first syringe barrel; 4—second syringe barrel; 5—buckle cover; 6—first rotating shaft; 7—pressing plate; 8—left housing; 9—first plunger; 10—second plunger; 11—handle; 12—supporting shaft; 13—first pre-tightening spring; 14—second rotating shaft; 15—screw; 16—right housing; 17—second pre-tightening spring; 18—power input structure; 181—arc-shaped rack portion; 182—first gear; 183—second gear; 184—lower pulley; 19—synchronous belt; 20—direction conversion structure; 201—driving helical gear; 202—upper pulley; 203—driven helical gear; 21—bracket; 22—first transmission shaft; 23—second transmission shaft; 24—sealing ring; 25—mixing blade.

### DETAILED DESCRIPTION OF EMBODIMENTS

In order to clearly understand the technical features, purposes, and effects of the disclosure, specific implementation methods of the disclosure are now explained with reference to the drawings.

### Embodiment 1

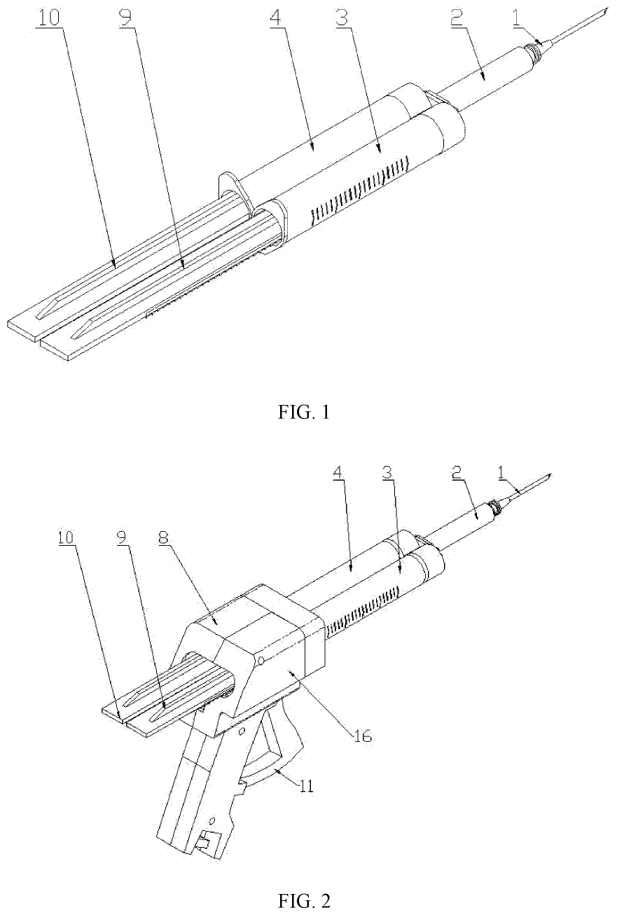

As shown in FIG. 1, the disclosure provides a multi-component mixing injection device. The device includes a mixer 2, an input end of the mixer 2 is provided with a first syringe barrel 3 and a second syringe barrel 4 arranged side by side, the first syringe barrel 3 is internally provided with a first plunger 9, the second syringe barrel 4 is internally provided with a second plunger 10, and an output end of the mixer 2 is provided with a needle 1. Different components in the first syringe barrel 3 and the second syringe barrel 4 are mixed by the mixer 2 and then ejected through the needle 1.

### Embodiment 2

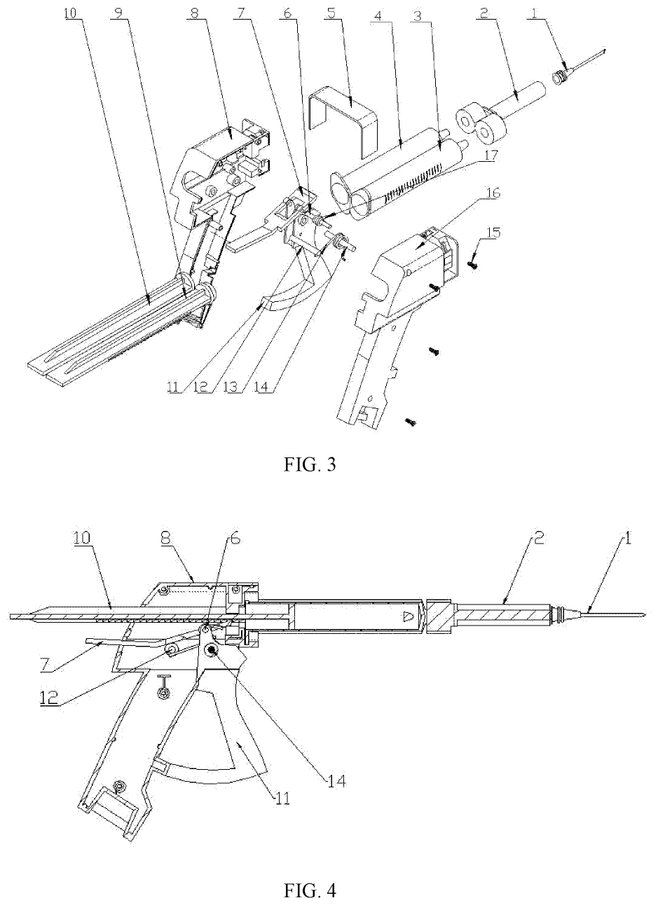

As shown in FIGS. 2-4, the disclosure provides a multi-component mixing injection device. The multi-component mixing injection device includes a mixer 2, an input end of the mixer 2 is provided with a first syringe barrel 3 and a second syringe barrel 4 arranged side by side, the first syringe barrel 3 is internally provided with a first plunger 9, the second syringe barrel 4 is internally provided with a second plunger 10, and an output end of the mixer 2 is provided with a needle 1.

The mixer 2 is a static mixer. The mixer 2 includes a static mixing tube, and a static element is arranged in the mixing tube. Different components in the first syringe barrel 3 and the second syringe barrel 4 are mixed when the different components flow through the static element, and the mixed components are ejected through the needle 1.

An end of the first syringe barrel 3 and an end of the second syringe barrel 4 are installed in a housing composed of a left housing 8 and a right housing 16. The left housing 8 and the right housing 16 are arranged oppositely, and the left housing 8 and the right housing 16 are locked by screws 15 after being buckled together. Ends of the left housing 8 and the right housing 16 near the first syringe barrel 3 and the second syringe barrel 4 are provided with a buckle cover 5, and the buckle cover 5 facilitates the installation of the first syringe barrel 3 and the second syringe barrel 4 in place.

A handle 11 is installed between the left housing 8 and the right housing 16, the handle 11 is hinged with the left housing 8 and the right housing 16 through a second rotating shaft 14, and the handle 11 can swing back and forth along the second rotating shaft 14. A first pre-tightening spring 13 is sleeved on the second rotating shaft 14, and the first pre-tightening spring 13 is configured to achieve automatic reset of the handle 11. An end of the first pre-tightening spring 13 is limited and fixed by a supporting shaft 12, the supporting shaft 12 is arranged between the left housing 8 and the right housing 16, and the supporting shaft 12 is configured to achieve pre-tightening of the first pre-tightening spring 13.

A top of the handle 11 is hinged with a pressing plate 7 through a first rotating shaft 6. During the swinging of the handle 11, the handle 11 drives the pressing plate 7 to move, thereby pushing the first plunger 9 and the second plunger 10 towards the first syringe barrel 3 and the second syringe barrel 4. A second pre-tightening spring 17 is sleeved on the first rotating shaft 6, and the second pre-tightening spring 17 is configured to achieve automatic reset of the pressing plate 7.

A side of the first plunger 9 and a side of the second plunger 10 near the pressing plate 7 are provided with serrations respectively, and a top of the pressing plate 7 is provided with a structure matched with the serrations. After the top of the pressing plate 7 meshes with the serrations, the pressing plate 7 is configured to drive the first plunger 9 and second plunger 10 to move. In addition, for each of the serrations, angles on two sides of the serration are different, an angle on a side of the serration is small and facilitates the pressing plate 7 moving back, and another angle of another side of the serration is large and facilitates preventing the movement of the pressing plate 7.

A corresponding working principle is as follows:
in the disclosure, the handle 11 is operated by one hand, and the handle 11 is configured to drive the first plunger 9 and the second plunger 10 to move towards the first syringe barrel 3 and the second syringe barrel 4 through the pressing plate 7. During the movement, different components in the first syringe barrel 3 and the second syringe barrel 4 are squeezed into the mixer 2 to achieve mixing, and after the mixing, the mixture is ejected from the needle 1.

### Embodiment 3

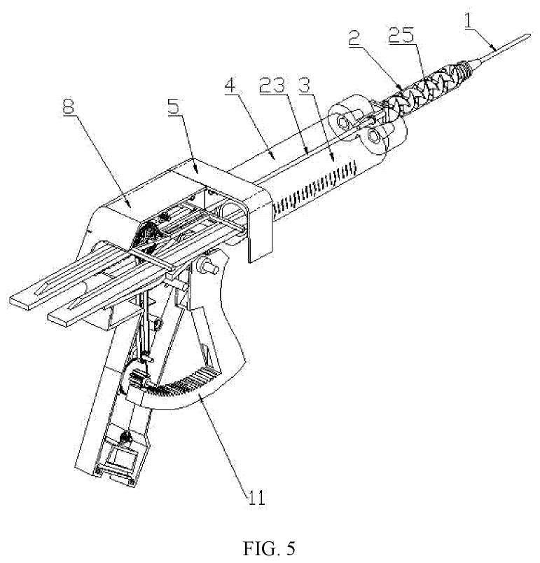

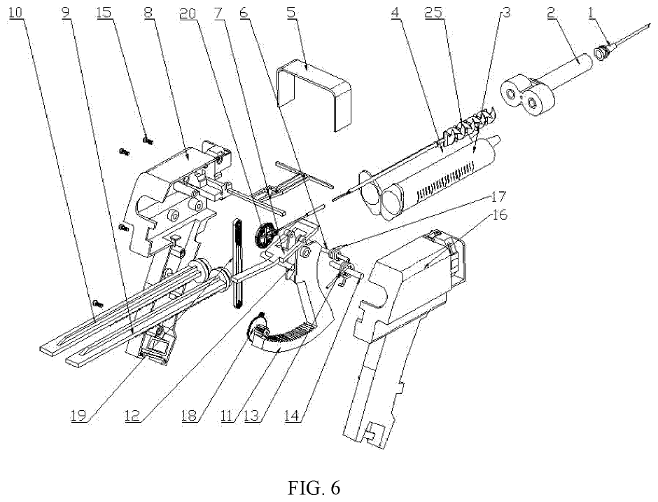

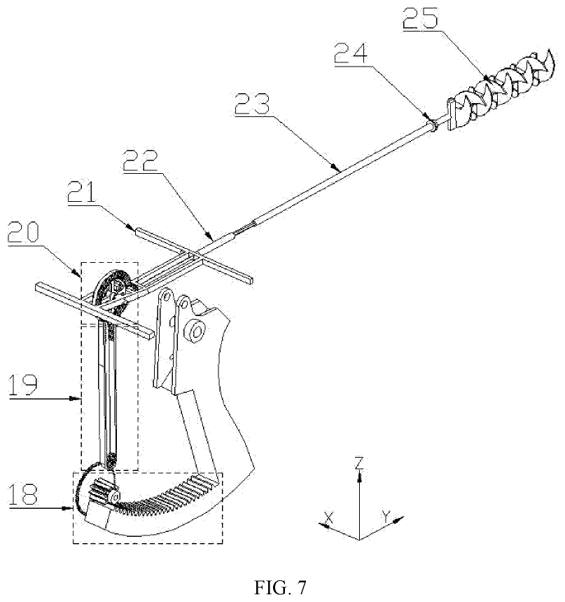

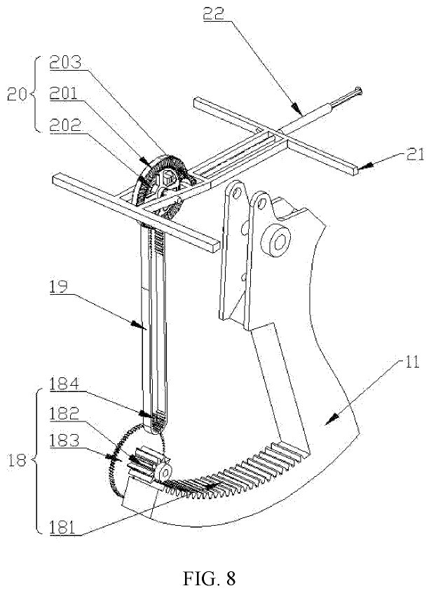

As shown in FIGS. 5-8, the disclosure provides a multi-component mixing injection device, differences between the device of the embodiment 3 and the device of the embodiment 2 are described as follows. In the device of the embodiment 3, preferably, each of an output end of the first syringe barrel 3 and an output end of the second syringe barrel 4 is an eccentric structure, and the eccentric structure facilitates discharging air from the needle 1. The mixer 2 is a dynamic mixer, the mixer 2 includes a dynamic mixing tube, and a dynamic mixing structure is configured to mix different components in the dynamic mixing tube.

The dynamic mixing structure includes a mixing component (for example, multiple mixing blades 25), the multiple mixing blades 25 are rotatably installed in the dynamic mixing tube, the multiple mixing blades 25 are installed on a power output end of a second transmission shaft 23. The second transmission shaft 23 is in a rotary sealing connection with the dynamic mixing tube through a sealing ring 24, and the sealing ring 24 is configured to ensure components in the mixer 2 will not leak and ensure the rotation of the second transmission shaft 23.

The second transmission shaft 23 is arranged above the first syringe barrel 3 and the second syringe barrel 4, and the second transmission shaft 23 is parallel with the first syringe barrel 3 and the second syringe barrel 4.

A power input end of the second transmission shaft 23 is connected to a power output end of the first transmission shaft 22. Preferably, the first transmission shaft 22 and the second transmission shaft 23 are arranged in a coaxial manner; furthermore, the first transmission shaft 22 may be connected to the second transmission shaft 23 through a coaxial structure or a universal coupling. A power input end of the first transmission shaft 22 is connected to a direction conversion structure 20. The direction conversion structure 20 is connected to a power input structure 18 through a synchronous belt 19.

The power input structure 18 includes an arc-shaped rack portion 181, and the arc-shaped rack portion 181 is arranged on the handle 11. The arc-shaped rack portion 181 meshes with a first gear 182, a side of the first gear 182 is provided with a second gear 183 arranged coaxially with the first gear 182, the second gear 183 meshes with a lower pulley 184, and the lower pulley 184 meshes with an inner surface of the synchronous belt 19.

The direction conversion structure 20 is configured to convert a rotational power vector in an X-axis direction to a rotational power vector in a Y-axis direction. The direction conversion structure 20 includes a driving helical gear 201 and a driven helical gear 203. The driving helical gear 201 meshes with the driven helical gear 203. The axis lines of the driving helical gear 201 and the driven helical gear 203 are perpendicular to each other. A side of the driving helical gear 201 is provided with an upper pulley 202 arranged coaxially with the driving helical gear 201, and the upper pulley 202 meshes with the inner surface of the synchronous belt 19. The driven helical gear 203 is installed on the power input end of the first transmission shaft 22. The driving helical gear 201 and the driven helical gear 203 are fixed in a housing composed of a left housing 8 and a right housing 16 through a bracket 21, and the bracket 21 is configured to ensure transmission accuracy of the device.

A corresponding working principle is as follows:
in the disclosure, the handle 11 is operated by one hand, and during the movement of the handle 11, the power input structure 18 generates rotational power. The rotational power is introduced to the direction conversion structure 20 above the synchronous belt 19 through the synchronous belt 19. The direction conversion structure 20 converts the rotational power in the X-axis direction into the rotational power in the Y-axis direction, thereby sequentially driving the first transmission shaft 22, the second transmission shaft 23, and the mixing blades 25 to rotate. During the movement of handle 11, the first plunger 9 and second plunger 10 are driven to move towards the first syringe barrel 3 and the second syringe barrel 4 through the pressing plate 7. During the movement of the first plunger 9 and second plunger 10, different components in the first syringe barrel 3 and the second syringe barrel 4 are squeezed into the mixer 2. The mixing blades 25 in the mixer 2 fully mix the different components, and mixed component are ejected from needle 1 after mixing.

Compared with the embodiment 2, the mixing method of in the embodiment 3 is no longer simple static mixing, but dynamic mixing similar to stirring. Under the same mixing efficiency, the size of the mixer 2 can be shortened to the maximum, and the shortened mixer 2 reduces the storage space and saves a mixing medium that needs to be filled in the storage space. At the same time, the shortening of the mixer 2 also improves the accuracy of operating a needle position with one hand.

The multi-component mixing injection device of the disclosure can be applied to treat chronic wounds (commonly known as ulcers). A proper amount of carboxymethyl cellulose (CMC) and aldehyded bletilla striata glucomannan (BSG) powder are taken as raw materials. The CMC and the aldehyded BSG powder are respectively dissolved with distilled water to prepare a CMC solution and an aldehyded BSG solution, the two solutions are mixed evenly with a volume ratio of 1:1 to prepare a CMC/aldehyded BSC mixed solution, and a resultant mass ratio of CMC to aldehyded BSC is 4:2. Ten milliliters (mL) of the CMC/aldehyded BSC mixed solution are injected into the first syringe barrel 3, umbilical cord blood platelet-rich plasma (PRP) into the second syringe tube 4, and a volume ratio of the umbilical cord blood PRP to the CMC/aldehyde BSG mixed solution is 1:1.

After the CMC/aldehyde BSG mixed solution and the umbilical cord blood PRP are mixed evenly in the mixer 2 to obtain a mixture, the first plunger 9 and the second plunger 10 are pushed to spray the mixture to an intended application area (i.e., a wound). The CMC/aldehyde BSG mixed solution serves as a weak activator and a cross-linking agent to make the umbilical cord blood PRP slowly solidify into a gel with good mechanical and adhesive properties within 10 minutes at 37 Celsius degrees (° C.), to completely cover the wound according to a shape of the wound.

The CMC, as a derivative of marine polysaccharides, cross-links with the aldehyde BSG, and the CMC and the aldehyde BSG together serve as the weak activator of the umbilical cord blood PRP.

The above are exemplary embodiments of the disclosure, and parts not detailed are common knowledge of ordinary those skilled in the art. The scope of protection of the disclosure is subject to the content of the claims, and any equivalent transformation based on the technical inspiration of the disclosure is also within the scope of protection of the disclosure.

What is claimed is:

1. A multi-component mixing injection device, comprising: a mixer (2); wherein an input end of the mixer (2) is provided with two or more syringe barrels arranged side by side, and each of the syringe barrels is internally provided with a plunger;
wherein an end of the syringe barrel is installed in a housing, and a handle (11) is installed to the housing and is configured to swing back and forth, a top of the handle (11) is hinged with a pressing plate (7) through a first rotating shaft (6), and the handle (11) is configured to drive the plunger to move towards the syringe barrel through the pressing plate (7); and
wherein the mixer (2) comprises a dynamic mixing tube, and a dynamic mixing structure is configured to mix different components in the dynamic mixing tube; the dynamic mixing structure comprises a plurality of mixing blades (25), the plurality of mixing blades (25) are installed in the dynamic mixing tube, the plurality of mixing blades (25) are installed on a power output end of a transmission shaft, and a power input end of the transmission shaft is connected to a direction conversion structure (20); the direction conversion structure (20) is connected to a power input structure (18); the power input structure (18) comprises an arc-shaped rack portion (181), and the arc-shaped rack portion (181) is arranged on the handle (11).

2. The multi-component mixing injection device as claimed in claim 1, wherein the transmission shaft comprises a first transmission shaft (22) and a second transmission shaft (23) arranged in a coaxial manner, a power output end of the second transmission shaft (23) is provided with the plurality of mixing blade (25), and the second transmission shaft (23) is in a rotary sealing connection with the dynamic mixing tube through a sealing ring (24).

3. The multi-component mixing injection device as claimed in claim 2, wherein the second transmission shaft (23) is arranged above the syringe barrels and is parallel with the syringe barrels; a power input end of the second transmission shaft (23) is connected to a power output end of the first transmission shaft (22); and a power input end of the first transmission shaft (22) is connected to the direction conversion structure (20).

4. The multi-component mixing injection device as claimed in claim 1, wherein the arc-shaped rack portion (181) meshes with a first gear (182), a side of the first gear (182) is provided with a second gear (183) arranged coaxially with the first gear (182), the second gear (183) meshes with a lower pulley (184), and the lower pulley (184) meshes with an inner surface of a synchronous belt (19).

5. The multi-component mixing injection device as claimed in claim 4, wherein the direction conversion structure (20) is connected to the power input structure (18) through the synchronous belt (19), the direction conversion structure (20) comprises a driving helical gear (201) and a driven helical gear (203); the driving helical gear (201) meshes with the driven helical gear (203); a side of the driving helical gear (201) is provided with an upper pulley (202) arranged coaxially with the driving helical gear (201), and the upper pulley (202) meshes with the inner surface of the synchronous belt (19); the driven helical gear (203) is installed on the power input end of the transmission shaft; and the driving helical gear (201) and the driven helical gear (203) are fixed in a left housing (8) and a right housing (16) through a bracket (21).

6. The multi-component mixing injection device as claimed in claim 1, wherein the housing comprises a left housing (8) and a right housing (16) arranged oppositely, the left housing (8) and the right housing (16) are locked by screws (15) after being buckled together; and ends of the left housing (8) and the right housing (16) near the syringe barrel are provided with a buckle cover (5).

7. The multi-component mixing injection device as claimed in claim 6, wherein the handle (11) is hinged with the left housing (8) and the right housing (16) through a second rotating shaft (14); a first pre-tightening spring (13) is sleeved on the second rotating shaft (14), the first pre-tightening spring (13) is configured to achieve automatic reset of the handle (11); a second pre-tightening spring (17) is sleeved on the first rotating shaft (6), and the second pre-tightening spring (17) is configured to achieve automatic reset of the pressing plate (7).

8. The multi-component mixing injection device as claimed in claim 1, wherein a side of the plunger near the pressing plate (7) is provided with a serration, and a top of the pressing plate (7) is provided with a structure matched with the serration.

9. The multi-component mixing injection device as claimed in claim 1, wherein an output end of the syringe barrel is an eccentric structure.

10. The multi-component mixing injection device as claimed in claim 1, an output end of the mixer (2) is provided with a needle (1).

11. A multi-component mixing injection device, comprising:
a needle (1);
a mixer (2), wherein an end of the mixer (2) is connected to the needle (1);
a first syringe barrel (3) and a second syringe barrel (4), wherein an end of the first syringe barrel (3) and an end of the second syringe barrel (4) are connected to the other end of the mixer (2);
a housing, arranged at the other ends of the first syringe barrel (3) and the second syringe barrel (4);
a first rotating shaft (6) and a second rotating shaft (14), arranged in the housing;
a pressing plate (7), arranged in the housing;
two plungers, arranged in the first syringe barrel (3) and the second syringe barrel (4) respectively;
a handle (11), hinged with the pressing plate (7) through the first rotating shaft (6) and hinged with the housing through the second rotating shaft (14), and configured to drive the two plungers to move towards the first syringe barrel (3) and the second syringe barrel (4) through the pressing plate (7) to squeeze different components in the first syringe barrel (3) and the second syringe barrel (4) into the mixer (2);
a power input structure (18), arranged on the handle (11);
a direction conversion structure (20), arranged in the housing, and connected to the power input structure (18) through a synchronous belt (19);
a mixing component, installed in the mixer (2); and
a transmission shaft, arranged on the first syringe barrel (3) and the second syringe barrel (4), wherein the transmission shaft is connected between the direction conversion structure (20) and the mixing component, and the handle is further configured to drive the power input structure (18) to move the direction conversion structure (20), and thus rotate the transmission shaft and the mixing component through the direction conversion structure (20) to mix the different components in the mixer (2).

12. The multi-component mixing injection device as claimed in claim 11, wherein the power input structure (18) comprises an arc-shaped rack portion (181), a first gear (182), a second gear (183), and a lower pulley (184); and
wherein the arc-shaped rack portion (181) meshes with the first gear (182), a side of the first gear (182) is provided with the second gear (183) arranged coaxially with the first gear (182), the second gear (183) meshes with to the lower pulley (184), and the lower pulley (184) meshes with an inner surface of the synchronous belt (19).

13. The multi-component mixing injection device as claimed in claim 12, wherein the direction conversion structure (20) comprises a driving helical gear (201), a driven helical gear (203), and an upper pulley (202).

14. The multi-component mixing injection device as claimed in claim 13, wherein the transmission shaft comprises a first transmission shaft (22) and a second transmission shaft (23) arranged in a coaxial manner.

15. A multi-component mixing injection device, comprising:
two syringe barrels, arranged side by side;
a mixer (2) comprising an input end and an output end, wherein the input end is connected to the two syringe barrels, and the output end is connected to a needle (1);
two plungers, matched with and connected to the two syringe barrels, respectively, and accommodated in a housing;
mixing blades (25), disposed in the mixer (2); and
a handle (11), connected to the housing and the mixing blades, wherein the handle is configured to drive the two plungers to move towards the two syringe barrels to make different components in the two syringe barrels squeeze into the mixer (2), and is further configured to rotate the mixing blades (25) to mix the different components in the mixer (2).
